(19) United States
(12) Patent Application Publication
(10) Pub. No.: US 2011/0236508 A1
(43) Pub. Date: Sep. 29, 2011

(54) L-ARGININE-BASED FORMULATION FOR ORAL ABSORPTION

(76) Inventors: Benzion Geshuri, Tel Mond (IL); Orna Levin, Kfar Neter (IL)

(21) Appl. No.: 13/131,869

(22) PCT Filed: Nov. 30, 2009
(86) PCT No.: PCT/IL2009/001126

§ 371 Date: May 30, 2011

(30) Foreign Application Priority Data
Nov. 30, 2008 (IL) 195602

(51) Int. Cl. A61K 33/08; A61K 31/198; A61K 38/06; A61K 38/07; A61K 38/08; A61K 36/16; A61K 31/555; A61P 9/10; A61P 1/08; A61Q 11/00; A61P 9/00; A61P 15/00

(52) U.S. Cl. 424/692; 514/565; 514/1.9; 424/752; 514/185; 514/562

(57) ABSTRACT

A formulation comprising large quantities of l-arginine and/or fat plaque dissolving agents which is palatable, stench free, and does not evoke nausea. The formulation is adapted to facilitate the adsorption of l-arginine to the blood system and to introduce high levels of l-arginine and/or other fat plaque dissolving agents such as EDTA, its derivatives or its salts into the blood system which are sufficient for effectively dissolving fat plaques in the artery. The formulation comprises at least 10% (w/w) L-arginine, edible organic acids, emulsifier(s), preservatives, flavorings, ethanol and water. Other embodiments may further include chromium salts, and EDTA or its derivatives and their salts

### BACKGROUND OF THE INVENTION

The present invention generally relates to dietary supplements and in particular to dietary supplement of fat plaque dissolving agents including L-arginine which serves as a sustained NO donor for vasodilation and inhibition of smooth muscle cell hyperprolifiration and improving endothel function providing preventative effects for the cardiovascular system and relief from symptoms.

Undesirable taste is one of several important formulation problems that are encountered with certain drugs and nutritional supplements. Several oral pharmaceuticals, numerous food and beverage products, and bulking agents have unpleasant, bitter-tasting components. The methods most commonly involved for achieving taste masking include various chemical and physical methods, which prevent the drug substance from interacting with taste buds. The simplest method involves use of flavor enhancers. Where these methods fail, more complex methodologies are adopted. Various techniques have been identified for taste-masking, including polymer coating, inclusion complex formation with cyclodextrin, use of ion exchange resins, solubility limiting methods, liposome, multiple emulsions, use of anesthetic agents, etc.

Flavor Enhancers are flavoring and perfuming agents, which can be obtained from either natural or synthetic sources. Natural products include fruit juices, aromatic oils such as peppermint and lemon oils, herbs, spices and distilled fractions of these. They are available as concentrated extracts, alcoholic or aqueous solutions, syrups or spirit. Use of flavor enhancers are limited only to unpleasant tasting substances, and is not applicable to oral administration of extremely bitter tasting drugs like that of L-arginine.

Micro-encapsulation, or Active Pharmaceutical Ingredient (API) particle coating, uses polymers or lipids to taste-mask bitter APIs. The coated composition may be incorporated into a great number of pharmaceutical formulations, including chewable tablets, effervescent tablets, powder and liquid dispersions.

Prepared micro-capsules of APIs with various cellulosic polymers have a pH-dependent solubility with the aim to mask its taste while assuring its release in the intestinal cavity. The drug release studies and the stability assay of the encapsulated moiety demonstrated microspheres represent a useful approach to achieve the proposed objectives. Low melting point substances, like lipophilic waxes, are also used for masking the bitter taste of the drugs. Such substances also have a deteriorating effect on the dissolution kinetics and, therefore, are not applicable to fast-disintegrating and fast-dissolving compositions.

The most widely used synthetic materials for coating are polymers such as polyacrylates referred to as Eudragit-types. Polymer coatings make it possible to formulate functional coatings, and thus to design solid pharmaceutical dosage forms with specific release profiles. Coating of small API particles has the disadvantage that due to the huge surface area of fine API particles, large quantities of polymeric coating material must be used. Typically, coating of very fine API crystals is associated with particle agglomeration. Breaking of these agglomerates could unleash the bitter taste again.

Cyclodextrins are cyclic oligosaccharides that form a toroid structure which has free hydroxyl groups pointing outward on its openings and a lypophylic cavity. Due to this arrangement cyclodextrins are capable of inclusion complex formation, which allows masking the bitter taste of drugs either by decreasing its solubility on digestion or decreasing the amount of drug particles exposed to taste buds, thereby reducing its perception of bitter taste. The use of cyclodextrins for taste-masking is limited because of very high concentrations necessary for taste masking.

Subsequent to its discovery, the endogenous arterial vasodilator “endothelium-derived relaxing factor” (EDRF) was determined to be chemically identical to the free radical gas nitric oxide (NO). Known to be present in macrophages, NO is thought to mediate a number of macrophage cytotoxic actions. Endogenous NO is believed to mediate many diverse physiological processes including vasorelaxation, immune responses, adhesion of leukocytes and platelets, and neurotransmission. The basic amino acid L-arginine is the precursor for the synthesis of NO in mammals.

As it can be synthesized endogenously from L-citrulline, L-arginine is classified as a non-essential amino acid in adults. However, in children and in conditions of accelerated growth as seen following trauma or infection, L-arginine synthesis may become inadequate. Thus, L-arginine may be considered “semi-essential” in certain situations. In addition to occurring in the liver, much of the endogenous synthesis of L-arginine from L-citrulline takes place in the proximal tubule of the kidney during the formation of urea. L-Arginine, which constitutes approximately 5% of the amino acid content of the typical adult diet, is absorbed in the lower ⅔ of the small intestine along with other basic amino acids. Absorption involves uptake by the gastrointestinal enterocytes, where about 60% of the L-arginine is metabolized, and only 40% reaches the systemic circulation intact. Although some protein-containing foods may have slightly higher L-arginine contents than others, nutritionally there is little difference between most proteins with respect to this amino acid. Thus the only possible way to effectively enhance the ingested Arginine in a person would be to supplement with the amino acid itself.

L-Arginine stimulates the secretion of a number of important hormones including hypothalamic corticotropin releasing factor (CRF), pituitary growth hormone and prolactin, pancreatic insulin, glucagon, pancreozymin and polypeptide, somatostatin, aldosterone, and adrenal catecholamines. L-Arginine given parenterally (30 g) is used to determine the ability of the pituitary to release growth hormone.

Nitric oxide formed from L-arginine appears to be present in all cells in the body and is believed essential in a number of important homeostatic processes. In blood NO is rapidly inactivated by oxyhemoglobin to form methemoglobin. While NO normally has a very short half-life of only 3-5 seconds, some of the NO formed in vivo can survive 30-40 times longer if reacted with nitroso adducts on albumin. Most of the biological effects of NO are mediated via the activation of soluble guanylyl cyclase which increases cyclic-GMP in cells. However, some NO-mediated effects are guanylyl cyclase independent.

The production of NO from L-arginine occurs by way of NO synthase (NOS). Three isoforms of NOS exist: inducible (iNOS), endothelial (eNOS), and neuronal (nNOS). Normally levels of L-arginine are in the millimolar range. As the Km (half saturation concentration) for NOS is in the micromolar range, one would predict that NOS should be saturated with its substrate L-arginine, and that additional L-arginine should not affect NO production. However, under various pathologic conditions L-arginine has been shown to increase NO and influence physiological function, and thus this “arginine paradox” has been the subject of much investigation. One explanation that has gained the most interest is the finding that high levels (2-10 times normal) of the endogenous L-arginine analog asymmetric dimethyl-L-arginine (ADMA) are present during many of these pathologic conditions and can inhibit NOS. The formation of ADMA is not from circulating free L-arginine, but appears to derive from the posttranslational methylation of peptide bound arginine in proteins (e.g., histones, heat shock proteins). Elevated ADMA, shown to be capable of inhibiting NOS, has been reported in renal failure, hypertension, preeclampsia, hypercholesterolemia, tobacco use, diabetes mellitus, and aging. Supplemental L-arginine is capable of competing with ADMA and overcoming this inhibitory effect. Elevations in cholesterol and associated atherogenic lipoproteins or glucose decrease the major catabolic enzyme involved in ADMA metabolism. This enzyme, dimethylarginine dimethylaminohydrolase (DDAH) is significantly decreased (40%-60%) in animals fed high-cholesterol diets or given streptozotocin which experimentally induces diabetes mellitus and hyperglycemia. The resulting disruption of ADMA metabolism by DDAH may explain the underlying dysregulation of NO synthesis in endothelial cells in various pathologic conditions.

An additional pathway involving the nonenzymatic synthesis of NO has been proposed, in which L-arginine combines with the highly reactive oxygen species hydrogen peroxide or superoxide anion to yield NO. This pathway may help to explain why L-arginine has been shown to be effective in some conditions characterized by oxidative stress.

Tissue injury and repair increases the demand for L-arginine. While initially there is a decrease in L-arginine and a corresponding increase in L-citrulline and NO in the injured tissue, during the repair period L-arginine continues to be depleted as L-ornithine production increases due to the action of arginase.

L-Arginine can undergo numerous metabolic fates. In addition to its role as a component of most proteins, this amino acid can be converted to urea, L-citrulline, L-ornithine, L-proline, L-glutamate, and polyamines such as putrescine. Creatine, the high-energy phosphate storage form found in skeletal muscles, is also formed from L-arginine. Recently the decarboxylation of L-arginine via L-arginine decarboxylase to form agmatine has been reported. Agmatine may act as an endogenous antihypertensive agent, similar in mechanism to that of clonidine.

L-arginine plays an important role in the body's response to injury. Within the cardiovascular system alterations in NO function have been linked to numerous diseases, many of which appear to originate in the vascular endothelial cells. A healthy vasculature is characterized by the presence of endothelial cell-produced, locally acting paracrine factors that favor vasodilatation, blood fluidity and inhibition of cell proliferation. In contrast, numerous cardiovascular disease states are characterized by an abundance of endothelial factors causing vasoconstriction, inflammation, thrombolytic activity and cell proliferation. The following table lists some of the factors that typically predominate in healthy and pathological blood vessels:

Endothelial Paracrine Factors Predominating in Healthy and
Diseased Blood Vessels

| FUNCTION | HEALTHY VESSELS | DISEASED VESSELS |
|---|---|---|
| Blood | Tissue plasminogen | von Willebrand factor |
| Fluidity | activator | |
| | Heparans | Adhesive glycoproteins |
| | Thrombomodulin | Plasminogen activator |
| | | inhibitor |
| Vascular | Nitric oxide | Endothelin |
| Tone | Natriuretic peptide | Angiotensin II |
| | Prostacyclin | |
| | Endothelium-derived | |
| | hyperpolarizing factor | |
| Cell | Nitric oxide | Insulin-like growth factor |
| Proliferation | Prostacyclin | Platelet-derived growth factor |
| | Transforming growth factor | Basic fibroblast growth factor |
| | beta | |

The delicate balance between these factors determines the overall health of the vasculature; thus, processes that can either augment or disrupt the synthesis, release, metabolism or actions of a particular paracrine factor may contribute to pathology or constitute an approach for a therapeutic intervention.

Atherosclerosis in experimental animals and in humans is associated with impaired vasodilation in response to normal physiological stimuli. Even patients at risk for the development of atherosclerosis, who have yet to demonstrate hypertension or other overt cardiovascular abnormalities, typically exhibit abnormal vasodilator responses when examined carefully. These abnormalities are thought to be partly due to an enhanced degradation of NO due to superoxide anion overproduction, reduced availability of the NOS cofactors, or an impaired synthesis of NO due to ADMA accumulation. While the degree of vasodilatory impairment is related to plasma low-density lipoprotein (LDL) levels and a number of other standard risk factors (e.g., smoking, hypertension, other hyperlipidemias), the ratio of L-arginine/ADMA in plasma may be the best correlated biochemical marker for predicting vascular dysfunction. Administration of L-arginine to experimental animals (hypercholesterolemic rabbits) and humans (hypercholesterolemic, hypertensive) has been shown to reverse the vasomotor dysfunction and restore vasodilatory responses to cholinergic agonists (e.g., methacholine, acetylcholine) that normally release EDRF. Studies in cultured endothelial cells also support these findings and the role of ADMA.

Exercise tolerance in experimental animals with compromised limb blood flow can be significantly improved by L-arginine administration. When administered a 6% L-arginine drinking solution, mice deficient in Apo-E, the apoprotein normally involved in the transport of cholesterol out of the circulation, showed a 61% increase in treadmill exercise performance. This beneficial effect of L-arginine was associated with an increase in overall NO synthesis, and could be blocked by a NOS inhibitor. Patients with peripheral arterial disease also benefit from L-arginine supplementation (8 g, bid, 14 days) as evidenced by a 150% improvement in walking distance. Additionally, patients with angina associated with coronary artery disease given L-arginine (9 g/day orally for 6 months) demonstrated improved acetylcholine-induced coronary vascular relaxation (149% improvement) with a 70% decrease in anginal episodes. L-Arginine (6 g/day) improved exercise tolerance in anginal patients and increased the time to 1 mm ST-segment depression during exercise-stress testing.

Platelet aggregation, leukocyte adhesion, proliferation of vascular smooth muscle and superoxide anion formation all enhance the formation of atherosclerotic plaques. NO inhibits these pro-atherogenic factors, and L-arginine administration reduces atherogenesis in a number of studies. Hypercholesterolemic rabbits and LDL receptor knockout mice both experience fewer intimal lesions when treated with L-arginine. On the other hand, administration of NOS inhibitors accelerates lesion formation. The potency of L-arginine in reducing lesion formation is similar to that observed with the HMG CoA reductase inhibitor lovastatin. Administration of L-arginine can lead to regression of preexisting intimal lesions in the hypercholesterolemic rabbit and can inhibit myointimal hyperplasia after balloon angioplasty.

Hypertension is characterized by endothelial dysfunction as evidenced by the finding that while vasodilator responses to endothelium-independent responses remain intact, such responses to endothelium-dependent processes (e.g., cholinergic agonist administration) are impaired, even in young patients just developing the disease. Administration of NOS inhibitors normally increases arterial blood pressure by 40% in experimental animals. While administration of NOS inhibitors to normotensive patients produces a similar hypertensive response, little response is observed in hypertensive patients following NOS inhibitor administration; this suggests the normally operating constant NO-mediated vasodilator tone is deficient in hypertension. Whether the primary defect in hypertension is endothelial function disruption or if the observed disruption is secondary to other pathologies is not known.

Several factors involved in the process of angiogenesis depend upon NO for their normal actions. Endothelial cells grown in culture and treated with proangiogenic factors (vascular endothelial growth factor, transforming growth factor beta, basic fibroblast growth factor) increase NO production, upregulate NOS, and are generally sensitive to inhibition by NOS inhibitors. Many effects of these angiogenic factors can be mimicked in vitro by administration of NO donors. In vivo administration of L-arginine (but not D-arginine) speeds healing and increases gastric blood flow in rats subjected to acid-induced ulcerations, a process known to require angiogenesis.

A number of disease states have been reported to respond beneficially to supplemental L-arginine. During sepsis and trauma, supplemental L-arginine improves nitrogen balance and reduces protein catabolism. In human breast cancer, supplementation with L-arginine increases the quantity and cytotoxic capability of lymphokine activated cells and natural-killer T-cells. L-Arginine has also been shown to be effective in a number of renally involved diseases such as nephrosclerosis associated with diabetes mellitus and progressive renal failure. Improvements in renal parameters such as glomerular filtration rate, renal blood flow and urinary protein excretion have been noted in experimental animal models for these disease states when L-arginine is administered. Improvements in renal function may also help enhance the clearance of ADMA and thus relieve the inhibition of NOS observed in some renal disorders.

L-Arginine Safety

In the few studies to date in which L-arginine HCl has been administered parenterally, metabolic acidosis and alterations in some electrolytes (e.g., potassium, phosphorus) have been noted. While these appear to occur with very large parenteral doses of the hydrochloride salt, these effects would not be anticipated following oral administration of more modest doses. Of the limited studies of L-arginine administered orally in humans, few report any adverse effects following acute or chronic treatment. A 1992 report that caused much concern demonstrated the ability of L-arginine (30 g/day, QID for 3 days) to double the rate of tumor protein synthesis in patients with breast cancer. However, studies prior and subsequent failed to replicate these findings, and in fact demonstrated a reduction in tumor growth. However, since the effect of L-arginine in patients with active malignancy is not fully known, caution should be exercised if L-arginine use is being considered in such patients.

Generally doses up to 30 g/day are well tolerated, the most common adverse effects (nausea and diarrhea) being reported infrequently. No changes in liver function, blood glucose, or plasma electrolytes have been noted. One study with 9 g/day reported one patient with a recurrence of oral herpes lesions, which resolved upon discontinuance of L-arginine treatment. In a recent study of 24 hypercholesterolemic patients administered 14 g/day for 12 weeks, no changes in plasma insulin, growth hormone or other serum chemistries or hematological parameters were noted except for a slight but clinically nonsignificant increase in blood urea nitrogen. There were no reports of any adverse effects in this study. L-Arginine (9 g/day) administered for 6 months in patients with angina appeared to be well tolerated with no adverse effects noted. Another study administered a medical food containing L-arginine (6.6 g/day, divided dose) for one week to 43 hypercholesterolemic patients. Beneficial effects on exercise tolerance and vasodilator responses were noted. The multicomponent product also contained vitamins B6, B12, C, E, folate and niacin in a soy protein base. It appeared highly effective, with no reports of adverse effects.

Available L-Arginine Products

L-Arginine is readily available in the form of dietary supplements in dosage of 100, 250, 500 mg capsules and tablets.

A product for L-arginine supplementation in the form of a medical food has been developed (HeartBar); it delivers L-arginine and a number of other antioxidants, vitamins and fiber. These other components have been shown to be of some benefit to patients with cardiovascular disease. A medical food is “formulated to be consumed or administered entirely under the supervision of a physician and is intended for the specific dietary management of a disease or condition for which distinctive nutritional requirements on the basis of recognized scientific principles are established by medical evaluation. Sold as a medical food, this product is intended to be used under a physician's supervision and generally would be expected to be of a higher pharmaceutical quality than dietary supplements.

Studies clearly demonstrate the potential of L-arginine supplementation to enhance NO-mediated cardiovascular health. The risk associated with L-arginine therapy appears minimal, except in a very small subset of patients (e.g., active malignancies, severe infection, diabetic retinopathy). Hence, several methods and vehicles are available for administering L-arginine.

U.S. Pat. No. 6,994,867 discloses a composition for inhibiting the narrowing of a blood vessel, comprising an oligomer of L-arginine, L-arginine, an L-arginine analog, or an L-arginine analog oligomer linked through a labile bond to a polymeric matrix, wherein the composition is used for coating an implantation device such as a stent.

U.S. Pat. No. 6,899,891 discloses a nutritional composition that delivers L-arginine among other amino acids in a combination that includes chromium complexes including chromium picolinate. The concentration of L-arginine is very low in this composition and the supplement ingredients of this composition are not adequate for increasing the relative amount of arginine

In U.S. Pat. No. 6,794,375 the inventors claim that by increasing the lipophilic nature of L-arginine significantly the unpalatable flavor associated with free forms of L-arginine and its derivatives is diminished. However this solution involves a synthetic step, the esterification of the free acid which is costly, and might introduce byproducts and contaminants in addition to the desire product. Furthermore, the digestibility of the esterified L-arginine products might not reach the levels of the free acid.

In order to achieve a pronounced effect of fat plaque dissolving by arginine, a daily dosage of 5-10 grams of l-arginine should be administered. L-arginine is available in the market in capsules of 500 mg, so in order to reach an intake of at least 5 grams per day, an inconvenient dosage of 10 capsules of 500 mg are needed. Furthermore, minerals that are present in the digestive system such as magnesium, calcium, and manganese to name a few, are responsible for complexation of L-arginine which form indigestible aggregates. L-arginine is therefore only partially adsorbed—only about 40% of l-arginine reaches the circulating system intact, therefore even a higher impractical dosage is required to reach effective levels of l-arginine.

One possible route to solve this problem would be to increase the absorbance of the substance into the body. It is common knowledge in the field that the best method to adsorb a substance into the body is through the mouth, and in particular—under the tongue. In the case of l-arginine, placing the substance under the tongue may come in the form of a tablet, a powder or a liquid suspension which contains it in a formula that overcomes the repulsive flavor and aroma of l-arginine. However, due to its extreme repulsive characteristics, such a formula does exist yet.

Furthermore, thus there is a long felt need to provide an L-arginine-based orally-obtained formulation which will contain an L-arginine compound in a concentration ranging from about 1% to about 80% (w/w) wherein the unpalatable flavor associated with free forms of L-arginine and its derivatives is diminished and even eliminated completely.

### SUMMARY OF THE INVENTION

It is one object of the present invention to provide an L-arginine-based orally-obtained formulation useful for dissolving fat plaques in arteries, said formulation comprising an L-arginine-containing compound in a concentration ranging from about 1% to about 80% (w/w);

wherein said formulation characterized by odor levels lower than 1 European Odor Units (ouE/m³) as obtained by olfactometry; and, further wherein the edibility of said formulation is at least 4 according to the arginine scale.

It is another object of the present invention to provide the formulation as defined above, wherein said formulation is characterized by pH of about 7 and, is palatable such that it is sustainable in a subject's mouth for at least 3 seconds allowing adsorption of at least 50% of said L-arginine content into said subject's blood system.

It is another object of the present invention to provide the formulation as defined above, wherein said formulation is characterized by pH of about 7 and, is palatable such that it is sustainable in a subject's mouth for at least 3 seconds allowing adsorption of about 0.5 gr to about 100 gr of said L-arginine content into said subject's blood system.

It is another object of the present invention to provide the formulation as defined above, wherein said formulation is capable of inducing nausea on a subject consuming at least 30 ml of said formulation per Kg weight of subject.

It is another object of the present invention to provide the formulation as defined above, additionally comprising:
a. an L-arginine-containing compound in a concentration ranging from about 1% to about 80% (w/w);
b. acids having an LD₅₀ (oral rabbit) greater than 2 gm/kg;
c. organic solvents having an LD₅₀ (oral rabbit) greater than 2 gm/kg;
d. flavoring additives capable of altering the taste of said formulation as sensed by a subject which consumes said formulation with respect to said taste without said flavoring additives;
e. sequestrants consisting of materials having a log_{(Ki)} greater than 2 in which i represents the coordination number of the complex which is an integer between 1 and 4, and K represents the stability coefficient of said complex;
f. metabolizable preservatives; and,
g. water.

It is another object of the present invention to provide the formulation as defined above, wherein at least one said L-arginine-containing compound is selected from the group consisting of L-arginine amino-acid, an oligo-peptide consisting of between two and ten L-arginine amino acids in which at least one amino-acid is L-arginine, a polypeptide consisting of at least ten amino-acids in which at least one amino-acid is L-arginine, derivatives of L-arginine which maintain the functionality of dissolving fat plaques in the artery, oligopeptides consisting of said derivatives of L-arginine, polypeptides consisting of said derivatives of L-arginine, and salts thereof.

It is another object of the present invention to provide the formulation as defined above, further comprising a buffer adapted to maintain said pH value at about 7.

It is another object of the present invention to provide the formulation as defined above, further comprising a sweetener.

It is another object of the present invention to provide the formulation as defined above, wherein at least one said sweetener is selected from the group comprising of sucrose, glucose, fructose, alkoxy aromatics, oximes, sulfamic acids, peptides, and succanilic acids, dihydro-chalcones and saccharin.

It is another object of the present invention to provide the formulation as defined above, wherein at least one of said acids is selected from a group consisting of phosphoric acid, nicotinic acid, citric acid, acetic acid, maleic acid, propionic acid, butyric acid, tartaric acid, fumeric acid, adipic acid, and lactic acid and any combination thereof.

It is another object of the present invention to provide the formulation as defined above, comprising between 1% and 80% (w/w) L-arginine containing compound.

It is another object of the present invention to provide the formulation as defined above, comprising between 10% and 40% (w/w) L-arginine containing compound.

It is another object of the present invention to provide the formulation as defined above, wherein at least one of said flavoring ingredient is selected from the group consisting of strawberry extract, pineapple extract, green tea leafs extract, anis extract, and combinations thereof.

It is another object of the present invention to provide the formulation as defined above, wherein said preservatives are selected from the group consisting of benzoate salt, nipagine, nipazole, nitrate salt, nitrite salt, propionate salt, sulphite salt, sulphor dioxide and combinations thereof.

It is another object of the present invention to provide the formulation as defined above, further comprising biocompatible emulsifiers, especially lecithin.

It is another object of the present invention to provide the formulation as defined above, further comprising at least one type of chromium complex.

It is another object of the present invention to provide the formulation as defined above, wherein said chromium complex is chromium picolinate.

It is another object of the present invention to provide the formulation as defined above, especially useful for oral treatments selected from a group consisting of plaque removal.

It is another object of the present invention to provide the formulation as defined above, especially useful for tooth bleaching, plaque remover, prevention of angina attacks, relieving intermittent claudication, balancing blood pressure, bad breath, improving endothelial function, sexual performance, sperm preparation or any combination thereof. It is another object of the present invention to provide the formulation as defined above, additionally comprising elements selected from a group consisting of Aspratic acid (Aspartate), Magnesium oxide (magnesia), Ginkgo Biloba, Chromium Picolinate, N-acetyl cysteine or any combination thereof.

It is another object of the present invention to a tablet comprising the formulation as defined above.

It is another object of the present invention to provide the tablet as defined above, additionally comprising reagents selected from a group consisting of citric acid, citric esters, nipagine, nipazole, arginine, glycerin, anis extract, ethanol, ascorbic acid, strawberry extract, chromium picolinate, EDTA calcium salt, l-lysine and combinations thereof.

It is another object of the present invention to provide a method for treating cardiovascular disorders. The method comprises steps selected inter alia from:
a. obtaining an l-arginine-based orally-obtained useful for dissolving fat plaques in the artery characterized by odor levels lower than 1 European Odor Units (ouE/m³) as obtained by olfactometry and wherein said formulation is capable of inducing nausea on a subject consuming at least 30 ml of said formulation per Kg weight of subject comprising an L-arginine-containing compound in a concentration ranging from about 1% to about 80% (w/w); wherein said formulation having a pH of about 7 is palatable such that it is sustainable in a subject's mouth for at least 3 seconds allowing adsorption of at least 70% of said L-arginine content into said subject's blood system comprising:
   i. an L-arginine-containing peptide comprising l-arginine in a concentration ranging from about 1% to about 80% (w/w);
   ii. acids which have an LD₅₀ (oral rabbit) higher than 2 gm/kg;
   iii. organic solvents, which have an LD₅₀ (oral rabbit) higher than 2 gm/kg;
   iv. flavoring additives;
   v. sequestrants consisting of materials having a log(Kᵢ)>2 in which i represents the coordination number of the complex which is an integer between 1 and 4, and K represents the stability coefficient of said complex;
   vi. preservatives; and,
   vii. water;
b. orally administering between about 5 ml to about 800 ml of said formula to a subject between once a week and five times a day; and,
c. repeating step (b) over a period of three days to twenty four months.

It is another object of the present invention to provide the method as defined above, additionally comprising step of selecting reagents from a group consisting of l-lysine and chromium complexes, especially chromium picolinate and combinations thereof.

It is another object of the present invention to provide the method as defined above, additionally comprising step of selecting said form of L-arginine-containing peptide from the group consisting of a monomer of l-arginine (amino acid), an oligopeptide consisting between two and ten l-arginine monomers, a polypeptide consisting of at least ten 1-arginine, derivatives of l-arginine and oligopeptides and polypeptides thereof.

It is another object of the present invention to provide the method as defined above, additionally comprising step of selecting at least one of said organic acids from a group consisting of phosphoric acid, nicotinic acid, citric acid, acetic acid, maleic acid, propionic acid, butyric acid, tartaric acid, fumeric acid, adipic acid, and lactic acid and any combination thereof.

It is another object of the present invention to provide the method as defined above, additionally comprising step of selecting said organic solvents from the group consisting of glycerin, glycerin derivatives, ethanol, phenol and combinations thereof.

It is another object of the present invention to provide the method as defined above, additionally comprising step of selecting said flavoring ingredients from the group consisting of mint, peppermint, vanilla, chocolate, strawberry extract, pineapple extract, green tea leafs extract, anis extract, and combinations thereof, and consist between 0.1% and 5% of the total weight of the formulation.

It is another object of the present invention to provide the method as defined above, additionally comprising step of selecting said sequestrants from the group consisting of EDTA, EDTA salts, especially EDTA calcium salt and EDTA sodium salt.

It is another object of the present invention to provide the method as defined above, additionally comprising step of selecting said flavoring ingredients from the group consisting of strawberry extract, pineapple extract, green or black tea leafs extract, anis extract, and combinations thereof.

It is another object of the present invention to provide the method as defined above, additionally comprising step of selecting said preservatives from the group consisting of benzoates, nitrates, nitrites, propionates, sulphites, sulphor dioxide and combinations thereof.

It is another object of the present invention to provide the method as defined above, wherein said formulation further comprising biocompatible emulsifiers, especially lecithin. It is another object of the present invention to provide the method as defined above, wherein said treatment is given prophylactically.

It is another object of the present invention to provide the method as defined above, wherein said subject is a mammalian.

It is still an object of the present invention to provide a method for producing an L-arginine-based orally-obtained formulation, comprising steps of:
a. mixing Arginine with Glycerin whilst heating said mixture with water;
b. neutralizing the acidity of said mixture by adding Citric Acid;
c. adding stabilizing egents selected from a group consisting of Nipagine and Nipazole or any combination thereof.

It is lastly an object of the present invention to provide the method as defined above, additionally comprising step of adding flavorings selected from a group consisting of Lemon, Strawberry, Orange, Mandarin or any combination thereof.

It is still an object of the present invention to provide the method as defined above, additionally comprising step of providing said L-arginine-based orally-obtained formulation with elements selected from a group consisting of Aspratic acid (Aspartate), Magnesium oxide (magnesia), Ginkgo Biloba, Chromium Picolinate, N-acetyl cysteine or any combination thereof.

It is still an object of the present invention to provide the formulation as defined above, especially used in mammals.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide means and methods for a formulation comprising large quantities of fat plaque dissolving agents, especially l-arginine which is palatable, stench free, and does not evoke nausea.

The term “mammal” herein refers to any mammal, especially a human, animal, dog, cat, horse cattle, mammalian household pets, and rodents

The term “about” refers herein to 10% more or less of the value which it refers to. The term “EDTA” refers hereinafter to ethylenediaminetetraacetic acid.

The term “oligomer” refers hereinafter to a molecule consisting of 2 to 10 covalently linked monomers.

The term “arginine scale” refers hereinafter to the scale as presented in example 1.

The term “edible” refers hereinafter to any substance that has an LD₅₀ (oral rat) higher than 2 g per Kg or LD₅₀ (oral mouse) higher than 2 g per Kg.

The term “ediblity” refers hereinafter to the ability to consume food, drug or specifically in the present invention the L-arginine based formulation as described in the present invention.

The term “pharmaceutically acceptable”, as used herein, means that the components present in the compositions of the present invention are compatible, safe, and suitable for oral administration to a mammal.

The term “European Odor Units” refers herein after as the odor concentration in an olfactometry testing procedure. In such testing, a diluted odorous mixture and an odor-free gas (as a reference) are presented separately from sniffing ports to a group of panelists, which are housed in an odor neutral room. They are asked to compare the gases emitted from each sniffing port, after which the panelists are asked to report the presence of odor together with a confidence level such as guessing, inkling, or certainty of their assessment. The gas-diluting ratio is then decreased by a factor of two (i.e. chemical concentration is increased by a factor of two). The panelists are asked to repeat their judgment. This continues for a number of dilution levels. The responses of the panelists over a range of dilution settings are used to calculate the concentration of the odor. The concentration is given in terms of European Odor Units (ouE/m³).

The most preferred embodiment of this invention is a formulation comprising at least 10% (w/w) L-arginine, edible organic acids, emulsifier(s), preservatives, flavorings, ethanol and water. Other embodiments may further include chromium salts, especially chromium picolinate, EDTA or its derivatives and their salts.

L-arginine can be formulated in the present invention in a variety of forms as long as these forms are readily metabolized in the body to provide free L-arginine amino acid. These forms include inter alia and in a non-limiting manner L-arginine salts, L-arginine amides, oligopeptides and polypeptides that contain L-arginine monomers further including oligo- or polypeptides that are only partially made of L-arginine and comprise other amino acids as well. Salts of L-arginine should be acceptable to be taken orally and may include inter alia arginine phosphate, arginine hydrochloride, arginine hydrobromide, arginine nicotinate, arginine citrate, arginine acetate, arginine maleate, arginine tartrate, arginine fumerate, arginine adipate, and arginine lactate. In case an L-arginine salt is used then the need to add an edible organic acid is eliminated.

The concentration of L-arginine or of the compound containing L-arginine in the formulations that are in the scope of this invention ranges between 1 mg/L and 800 g/L thus providing a full scale of products for various types of treatment. Most preferably the concentrations range between 100 g/L and 400 g/L.

An edible organic acid is needed to be added to the formulation for the neutralization of the alkaline L-arginine. The edible organic acids can be selected from the group including phosphoric acid, nicotinic acid, citric acid, acetic acid, maleic acid, propionic acid, butyric acid, tartaric acid, fumeric acid, adipic acid, and lactic acid. The most preferred edible organic acid to be formulated in this invention is citric acid.

The formulation is set to be at a pH allowing optimal absorbance of L-arginine through the mouth, which we found to be between pH=6 and pH=8 and most preferably at about pH=7. For this reason the amount of edible organic acid to be added is set to bring the pH of the formulation to the above mentioned range.

The flavoring ingredients that can be added to the formulation are inter alia mint, peppermint, vanilla, chocolate, strawberry extract, pineapple extract, green tea leafs extract, anis extract, and combinations thereof, in acceptable amounts that are sufficient to be sensed by an average person. These materials are included at levels of from about 0.1% to about 5% (w/w), preferably from about 0.1% to about 1% (w/w), and most preferably from about 0.25% to about 0.75% (w/w)

According to one embodiment of the present invention is providing the component in a sweetener which is included in an amount of from about 0.001% to about 5% (w/w), preferably from about 0.01% to about 1% (w/w), and most preferably from about 0.025% (w/w) to about 0.5% (w/w). Examples of useful sweeteners include but are not limited to, standard natural sweeteners, such as sucrose, glucose, and fructose synthetic alkoxy aromatics, such as Dulcin and P-4000; synthetic oximes, such as perilartine; synthetic sulfamic acids, such as acesulfame; peptides, such as aspartyl malonates and succanilic acids; dihydro-chalcones, Sucralose, glucose and, most preferably, saccharin (o-benzoic sulfimide).

The flavoring ingredients that can be added to the formulation are inter alia benzoates, nitrates, nitrites, propionates, sulphites, sulphor dioxide and combinations thereof.

Preservatives that can be added to the formulation are inter alia benzoate salt, nipagine, nipazole, nitrate salt, nitrite salt, propionate salt, sulphite salt, sulphor dioxide and combinations thereof. These ingredients can be added in pharmaceutically acceptable amounts in order to prolong and enhance the shelf life of the formulation.

The compositions of the present invention may also optionally contain other components conventionally found in food or pharmaceutical compositions, in their art-established levels of use. Examples of such components include binders, bulking agents, emulsifiers such as lecithin, vitamins, minerals, anti-oxidants, starches, flour, milk or milk extracts, such as lactose, sweeteners or flavorants not falling within the definitions given above, vegetable proteins, protein hydrosylates, microbial proteins, yeast extracts, gelatin, vegetable gums, cocoa, chocolate, colorants, and mixtures of the foregoing.

The formulation embodiments of this invention are useful for treating cardiovascular disorder by orally administering to a subject between about 5 ml and about 80 ml or 5-10 grs. At this period of treatment reduction of fat plaque in the arteries is significant and easily measurable nonetheless the treatment may be extended indefinitely without causing any harm to the subject for prophylactic purposes.

According to another embodiment of the present invention, the L-arginine-based orally-obtained formulation is used in urology treatments.

According to another embodiment of the present invention the L-arginine-based orally-obtained formulation is used for oral treatments selected from a group consisting of plaque removal.

According to another embodiment of the present invention, the L-arginine-based orally-obtained formulation is used for balancing blood pressure.

According to another embodiment of the present invention, the L-arginine-based orally-obtained formulation is used for prevention of angina attacks and relieving intermittent claudication.

According to another embodiment of the present invention, the L-arginine-based orally-obtained formulation is used for sexual performance, sperm preparation.

According to another embodiment of the present invention, the L-arginine-based orally-obtained formulation additionally comprising elements selected from a group consisting of Aspratic acid (Aspartate), Magnesium oxide (magnesia), Ginkgo Biloba, Chromium Picolinate, N-acetyl cysteine or any combination thereof.

It should be noted that when N-acetyl cysteine is added to the L-arginine-based orally-obtained formulation, said formulation functions as anti-oxidant since N-acetyl cysteine contains a thiol group (namely sulfur and hydrogen atoms, —SH).

### EXAMPLES

The following examples are presented to further illustrate this invention. The examples are intended in an illustrative sense and not a limitative sense. The invention includes the embodiments shown and described herein and equivalents thereof.

### Example 1

### Evaluation of Taste and Sensory Attributes

Compositions within the scope of the present invention were prepared by dissolving arginine in water to form solutions comprising 15% by weight arginine. The pH of the solutions were adjusted from the original pH of approximately 10 to various pHs ranging from pH 3 to pH 8 using concentrated phosphoric acid. The solutions were then evaluated for taste and sensory attributes and rated according to the following arginine scale:

The Arginine Scale:

| Not edible | | | | Fully acceptable |
|---|---|---|---|---|
| 1 | 2 | 3 | 4 | 5 |

It should be emphasized that the arginine scale is composed of 1 to 5 stages. In which 5 is fully acceptable; and, 1 is not edible.

The solutions were evaluated by 19 volunteers (man and female of different ages ranging from 25 to 80 years old). The results of the evaluation are presented in Table I below:

| | | | | | | Arginiline |
|---|---|---|---|---|---|---|
| | | | | | | according to the |
| | pH 5 | pH 7 | pH 7 H2O | pH 8 | pH 4 | present invention |
| Male | 4 | 5 | 4 | 4 | 3 | 5 |
| Male | 2 | 3 | 2 | 3 | 2 | 4 |
| Male | 1 | 3 | 2 | 3 | 1 | 4 |
| Male | 2 | 4 | 5 | 5 | 1 | 4 |
| Male | 2 | 2 | 1 | 3 | 1 | 4 |
| Male | 1 | 3 | 2 | 1 | 1 | 2 |
| Male | 3 | 5 | 4 | 4 | 1 | 5 |
| Male | 2 | 3 | 1 | 1 | 1 | 3 |
| Male | 1 | 3 | 3 | 4 | 1 | 4 |
| Male | 1 | 5 | 1 | 2 | 1 | 5 |
| Female | 1 | 4 | 1 | 3 | 1 | 4 |
| Female | 1 | 3 | 2 | 4 | 1 | 4 |
| Female | 1 | 4 | 1 | 1 | 3 | 3 |
| Female | 1 | 4 | 1 | 1 | 1 | 5 |
| Female | 2 | 3 | 1 | 1 | 1 | 3 |
| Female | 1 | 3 | 4 | 5 | 1 | 5 |
| Female | 1 | 2 | 1 | 2 | 1 | 3 |
| Female | 1 | 3 | 1 | 1 | 1 | 3 |
| Female | 3 | 1 | 1 | 1 | 3 | 5 |
| AVG. | 2 | 3 | 2 | 3 | 1 | 4 |
| Total score | 31 | 63 | 38 | 49 | 26 | 75 |

It should be emphasized that the solution titled ‘pH 7’ is a solution containing Arginine and the solution titled ‘pH 7 H2O’ is a solution without Arginine.

The results demonstrate that at acidic pH below 7 the taste and sensory attributes of arginine is significantly improved. As is clearly demonstrated in the table, the Arginiline solution according to the present invention received both:

1. The highest average scores (4 out of 5); and,

2. The highest total score (75 out of max 95);

### Example 2

### Use in Doppler's Examination of the Carotid Arteries

The L-arginine-based formulation according to the present invention was used in 4 different Doppler's test of the carotid arteries. As is commonly known, said test indicates the passage status of blood to the brain through the carotid arteries.

A 67 years old male patient who suffered from 50-69% obstruction in the right carotid artery was administered regularly the L-arginine-based formulation for a period of 7 months. After 7 months the obstruction had decreased to about 20-40%. After a period of another 6 months the patient was examined and there was no difference in the obstruction. The following table, table 1, illustrates the first Carotid Doppler exam (showing the 50-69% obstruction). Table 1 also illustrates the Peak Systolic Velocity (PSV), the End Diastolic Velocity (EDV) and the carotid Index (denotes in table 1 as PSV ICA/CC).

TABLE 1
First Carotid Doppler exam
CAROTID DOPPLR

| Diameter | PSV | EDV | PSV |
|---|---|---|---|
| stenoses | [cm/sec] | [cm/sec] | ICA/CCA |
| <50% | <125 | >40 | <2 |
| 69%-50% | 230-125 | 100-40 | 4.0-2.0 |
| 95%-70% | >230 | >100 | >4 |

The following table, table 2, illustrates the Flow Velocity—of the right carotid artery:

TABLE 2
Flow Velocity
Flow Velocity (cm/sec)

| | | |
|---|---|---|
| CCA | ICA | ECA |

| PSV | EDV | PSV | EDV | PSV | EDV |
|---|---|---|---|---|---|
| 71 | 14 | 139 | 53 | 42 | 7 |

The following table, table 3, illustrates the PSV divided by EDV of the right carotid artery after the patient has taken the L-arginine-based formulation for about 7 months.

TABLE 3
PSV/EDV
PSV/EDV

| right | carotid |
|---|---|
| artery | cm/sec |
| 74 | CCA PROX |
| 82 | CCA DIST |
| 90 | ECA |
| 58 | ICA BIF |
| 107 | ICA PROX |
| 110 | ICA DIST |
| 33 | VERT |

As can be seen from tables 2 and 3, there was an improvement (i.e., decrease) in the flow rate of the ICA from 139 PSV to 107 in the ICA prox or even to 52 in the ICA bif.

The following table, table 4, illustrates the PSV divided by EDV of the right carotid artery after the patient has taken the L-arginine-based formulation for about a year and a month.

TABLE 4
PSV/EDV
PSV/EDV

| right | carotid |
|---|---|
| artery | cm/sec |
| 62 | CCA |
| | PROX |
| 76 | CCA DIST |
| 71 | ECA |
| 50 | ICA BIF |
| 117 | ICA PROX |
| 100 | ICA DIST |
| 50 | VERT |

As can be seen from tables 3 and 4 there is difference in the obstruction (the flow rate of the ICA bif is still about 50 and the ICA prox is still about 110).

### Example 3

### Use as Tooth Bleaching

According to another embodiment of the present invention, the formulation as provided by the present invention can be used as tooth bleaching.

Compositions within the scope of the present invention were prepared by dissolving arginine in water to form solutions comprising 15% by weight arginine.

The solutions were then evaluated as for their tooth bleaching/tooth whitening functioning according to the following tooth color's scale:

| White | | | | | | | | Yellow | |
|---|---|---|---|---|---|---|---|---|---|
| 1 | 2 | 3 | 4 | 5 | 6 | 7 | 8 | 9 | 10 |

4 patients (2 males; 2 females) in the ages of 25-57 were tested.

The following table provides the initial data concerning the tooth's color prior to the administration of the composition of the present invention:

| patient | tooth color |
|---|---|
| male | 9 |
| female | 8 |
| male | 8 |
| female | 7 |
| AVG. | 8 |

The average color was 8 according to the above mentioned scale.

The following table provides the initial data concerning the tooth's color after the administration of the composition of the present invention for a few days (max a week):

| patient | tooth color |
|---|---|
| male | 3 |
| female | 2 |
| male | 2 |
| female | 2 |
| AVG. | 2.25 |

As can be seen from the table—the tooth's color changed from an average of 8 to an average of 2-3 within a few days (till max a week) of using the above mentioned composition.

It should be emphasized that the same composition was also used as ointment (paste) and the same results were obtained.

The test was repeated using several indicators e.g., methylene blue, dye indicator, erythrosine, FDC, brilliant blue or any combination thereof.

### Example 4

### Use as Plaque Remover from the Teeth

According to another embodiment of the present invention, the formulation as provided by the present invention can be used as plaque remover.

Compositions within the scope of the present invention were prepared by dissolving arginine in water to form solutions comprising 15% by weight arginine.

2 patients in the ages of 52, 57 were tested for removing plaque.

The results demonstrated that plaque in the size range of less than 2 milimetters were successfully removed in 3-5 weeks time.

Bigger sized plaque have been partially resolved.

The indicator that was used was methylene blue.

A second experiment was conducted in which 6 patients (4 females and 2 males) were tested for removing plaque. First 3CC of methylene blue (as an indicator) was given and the patient were instructed to rub said indicator on the tongue. Next, 5 CC of the above mentioned arginine based solutions were given to each of the patients. The patients were told to gurgle said solution for 30 seconds and extract said solution from said mouth. The results revealed an improvement of about 30-50% in ease of plaque removing.

Such use was also demonstrated in mammals namely, domestic animals, namely dogs, cats. A Labrador retriever typed dog was given about 10 cc of the above mentioned arginine solution for about 5 weeks. The arginine solution was sprayed and dripped on the Labrador retriever's food once a day. The tooth's color of said Labrador was initially scaled as level 8 on the tooth color's scale.

After said period of 5 weeks the Labrador retriever's teeth were whitened and scaled as level 1-2 on the tips of the teeth and level 4-5 on the basis of the teeth.

The same experiment was conducted in 2 boxers typed dogs with similar success.

It should be pointed out that said arginine solution had also neutralized bad breath,

According to another embodiment of the present invention, the formulation is used as paste or as mouth water having concentration ranging from 15% to 45% by weight arginine.

It should be emphasized that the same composition was also used as ointment (paste) and the same results were obtained.

According to another embodiment of the present invention, the above mentioned arginine solution is also useful for treating bad breath.

### Example 5

### Improvement in Endothelial Function

According to another embodiment of the present invention, the L-arginine-based formulation is used for improvement in endothelial function.

According to recent studies (e.g., Mullen M J, Wright D, Donald A E, Thorne S, Thomson H, Deanfield J E: Atorvastatin but not L-arginine improves endothelial function in type I diabetes mellitus: a double-blind study. J Am Coll Cardiol 36:410-416, 2000; Amir Lerman, MD; John C. Burnett, Jr, MD; Stuart T. Higano, MD; Linda J. McKinley, RN; David R. Holmes, Jr, MD, Long-term L-Arginine Supplementation Improves Small-Vessel Coronary Endothelial Function in Humans, Circulation. 1998; 97:2123-2128; Bode-Böger S M, Muke J, Surdacki A, Brabant G, Böger R H, Frölich J C, Oral L-arginine improves endothelial function in healthy individuals older than 70 years, Vasc Med. 2003 May; 8(2):77-81), L-Arginine formulation can be used for improving endothelial function.

As is well known, coronary endothelial dysfunction is characterized by an imbalance between endothelium-derived vasodilating and vasoconstricting factors and coronary vasoconstriction in response to the endothelium-dependent vasodilator acetylcholine. Amir Lerman's randomized study was designed to test the hypothesis that long-term, 6-month supplementation of L-arginine, the precursor of the endothelium-derived vasodilator NO, reverses coronary endothelial dysfunction to acetylcholine in humans with nonobstructive coronary artery disease.

In Lerman's study, Twenty-six patients without significant coronary artery disease on coronary angiography and intravascular ultrasound were blindly randomized to either oral L-arginine or placebo, 3 g TID.

Endothelium-dependent coronary blood flow reserve to acetylcholine (10⁻⁶ to 10⁻⁴ mol/L) was assessed at baseline and after 6 months of therapy.

There was no difference between the two study groups in clinical characteristics or in the coronary blood flow in the response to acetylcholine at baseline.

After 6 months, the coronary blood flow in response to acetylcholine in the subjects who were taking L-arginine increased compared with the placebo group (149±20% versus 6±9%, P<0.05). This was associated with a decrease in plasma endothelin concentrations and an improvement in patients' symptoms scores in the L-arginine treatment group compared with the placebo group.

Thus, it is shown that long-term oral L-arginine supplementation for 6 months in humans improves coronary small-vessel endothelial function in association with a significant improvement in symptoms and a decrease in plasma endothelin concentrations.

Therefore, according to another embodiment of the present invention, L-arginine can be used as a therapeutic composition for patients with coronary endothelial dysfunction and nonobstructive coronary artery disease.

Such functioning was also demonstrated by Bode-Böger SM's article, in which he demonstrated whether oral L-arginine, the substrate for NO synthesis, can improve impaired Flow-mediated dilation (FMD) in healthy very old people.

As is known, Ageing is associated with progressive endothelial dysfunction in normal humans. FMD of the brachial artery is impaired in elderly individuals with cardiovascular disease and vascular nitric oxide (NO) bioavailability is reduced.

In Bode-Böger SM's double-blind, randomized crossover trial, 12 healthy old subjects (age 73.8+/−2.7 years) took L-arginine (8 g p.o. two times daily) or placebo for 14 days each, separated by a wash-out period of 14 days.

FMD was determined by high-resolution ultrasound in the brachial artery during reactive hyperaemia. Baseline artery diameter was 3.88+/−0.18 mm. L-Arginine significantly improved FMD (to 5.7+/−1.2%, p<0.0001), whereas placebo had no effect (−0.25+/−0.7%; n.s.). After L-arginine, plasma levels of L-arginine increased significantly (114.9+/−11.6 versus 57.4+/−5.0 micromol/l), but placebo had no effect. As NO synthesis can be antagonized by its endogenous inhibitor asymmetric dimethyl L-arginine (ADMA), we determined ADMA plasma concentrations, which were elevated at baseline in comparison to healthy middle-aged individuals (3.9+/−0.2 versus 1.0+/−0.1 micromol/l; p<0.0001). ADMA remained unchanged during treatment, but L-arginine supplementation normalized the L-arginine/ADMA ratio (p<0.05).

This concludes that in healthy very old age endothelial function is impaired and may be improved by oral L-arginine supplementation, probably due to normalization of the L-arginine/ADMA ratio.

According to another embodiment of the present invention means and method for avoiding L-lysine deficiency in Arg-enriched diet are provided. It was found that subscribing L-arginine to patients, namely patients suffering from herpes, may result in the bio-synthesis of other amino acids in the body, namely L-lysine.

Thus, according to another embodiment of the present invention, an L-arginine-based formulation adapted not to damage (permanent of temporary) the bio-synthesis (namely, the natural, body synthesis) of amino-acids and their derivatives, namely L-lysine is provided.

It is another object of the present invention to provide an L-arginine-based formulation which comprises ingredients, adapted to prevent any damage the synthesis of amino-acids and their derivatives, namely L-lysine.

It is another object of the present invention to provide an L-arginine-based formulation comprising an appropriate amount of amino-acids and their derivatives, namely L-lysine.

### REFERENCES

1. Reyes A A, Karl I E, Klahr S. Role of arginine in health and in renal disease. Am J. Physiol 1994; 267: F331-F346.
2. Usui M, Matsuoka H, Miyazaki H, Ueda S, Okuda S, Imaizumi T. Increased endogenous nitric oxide synthase inhibitor in patients with congestive heart failure. Life Sci. 1998; 62(26):2425-2430.
3. Boger R H, Bode-Boger S M, Sxuba A, Tsao P S, Chan J R, Tangphao O, Blaschke T F, Cooke J P. ADMA: a novel risk factor for endothelial dysfunction: its role in hypercholesterolemia. Circulation 1998; 96S: A173.
4. Matsuoka H, Itoh S, Kimoto M, Kohno K, Tamai O, Wada Y, Yasukawa H, Iwami G, Okuda S, Imaizumi T. Asymmetrical dimethylarginine, an endogenous nitric oxide synthase inhibitor, in experimental hypertension. Hypertension 1997; 29:242-247.
5. Nagase S, Takemura K, Ueda A. A novel nonenzymatic pathway n for the generation of nitric oxide by the reaction of hydrogen peroxide and D- or L-arginine. Biochem Biophys Res Commun 1997; 233:150-153.
6. Ito A, Tsao P S, Adimoonlam S, Kimoto M, Ogawa T, Cooke, J P. A novel mechanism for endothelial dysfunction: dysregulation of dimethylarginine dimethylaminohydrolase. Circulation 1999; 99:3092-3095.
7. Albina J E, Mills C D, Barbul A, Thirkill C E, Henry W L Jr, Mastrofrancesco B, Caldwell MD. Arginine metabolism in wounds. Am J Physiol 1988; 254:E459-E467.
8. Raeger M A, Cooke J P, Mendelsohn M E, Gallagher S J, Coleman S M, Loscalzo J, Dzau V J. Impaired vasodilatation of forearm resistance vessels in hypercholesterolemic humans. J Clin Invest 1990; 86:228-234.
9. Celermajer D S, Sorensen K E, Gooch V M, Speigelhalter D J, Miller O W, Sullivan I D, Lloyd J K, Deanfield J E. Non-invasive detection of endothelial dysfunction in children and adults at risk for atherosclerosis. Lancet 1992; 340:1111-1115.
10. Vallance P, Leone A, Calver A, Collier J, Moncada S. Endogenous dimethylarginine as an inhibitor of nitric oxide synthesis. J Cardiovasc Pharmacol 1992; 20 (Suppl 12):560-562.
11. Craeger M A, gallagher S J, Girerd X J, Coleman S M, Dzau, V J, Cooke J P. L-Arginine improves endothelium-dependent vasodilation in hypercholesterolemic humans. J Clin Invest 1992; 90:1248-1253.
12. Cooke J P, Andon N A, Girerd X J, Hirsch A T, Creager M A. Arginine restores cholinergic relation of hypercholesterolemic rabbit thoracic aorta. Circulation 1991; 83:1057-1062.
13. Azumi H, Sato J, Hamasaki H, Sugimoto A, Isotani E, Obayashi S. Accumulation of endogenous inhibitors for nitric oxide synthesis and decreased content of L-arginine in regenerated endothelial cells. Br J Pharmacol 1995; 115:1001-1004.
14. Maxwell A J, Schauble E, Bernstein D, Cooke J P. Limb blood flow during exercise is dependent upon nitric oxide. Circulation 1998; 98:369-364.
15. Boger R H, Bode-Boger S M, Thiele W, Creutzig A, Alexander K, Frolich J C. Restoring vascular nitric oxide formation by L-arginine improves the symptoms of intermittent claudication in patients with peripheral arterial occlusive disease. J Am Coll Cardiol 1998; 32:1336-1344.
16. Lerman A, Burnett J C Jr, Higano S T, McKinley L J, Holmes D R Jr. Long-term L-arginine supplementation improves small-vessel coronary endothelial function in humans. Circulation 1998; 97:2123-2128.
17. Ceremuzynski L, Chamiec T, Herbaczynska-Cedro K. Effect of supplemental oral L-arginine on exercise capacity in patients with stable angina pectoris. Am J Cardiol 1997; 80:331-333.
18. Boger R H, Bode-Boger S M, Brandes R P. Dietary L-arginine reduces the progression of atherosclerosis in cholesterol-fed rabbits: comparison with lovastatin. Circulation 1997; 96:1282-1290.
19. Candipan R C, Wang B, Buitrago R, Tsao P S, Cooke J P. Regression or progression: dependency on vascular nitric oxide. Arterioscler Vasc Biol 1996; 16:44-50.
20. Schwarzacher S P, Lim T T, Wang B Y, Kernoff R S, Niebauer J, Cooke J P, Yeung A C. Local intramural delivery of L-arginine enhances nitric oxide generation and inhibits lesion formation after balloon angioplasty. Circulation 1997; 95:1863-1869.
21. Cooke J P, Dzau V J. Nitric oxide synthase: role in the genesis of vascular disease Annu Rev Med 1997; 48:489-509.
22. Calver A, Collier J, Moncada S, Vallance P. Effect of local intraarterial NG-monomethyl-L-arginine in patients with hypertension: the nitric oxide dilator mechanism appears abnormal. J Hypertens 1992; 10:1025-1031.
23. Brzozowski T, Konturek S J, Sliwowski Z, Drozdowicz D, Zaczek M, Kedra D. Role of L-arginine, a substrate for nitric oxide-synthase, in gastroprotection and ulcer healing. J. Gastroenterol 1997; 32:442-452.
24. Brittenden J, Heys S D, Miller I, Sarkar T K, Hutcheon A W, Needham G, Gilbert F, McKean M, Ah-See A K, Eremin O. Dietary supplementation with L-arginine in patients with breast cancer (>4 cm) receiving multimodality treatment: report of a feasibility study. Br J Cancer 1994; 69:918-921.
25. Sanders P W. L-Arginine and arginine analogs in progressive renal failure. Blood Purif 1995; 13:219-227.
26. Klahr S. Can L-arginine manipulation reduce renal disease? Sem Nephrol 1999; 19:304-309.
27. Park G M, Heys S D, Blessing K, Kelly P, McNurlan M A, Eremin O, Garlik P J. Stimulation of human breast cancers by dietary L-arginine. Clin Sic 1992; 82:413-417.
28. Tachibana K, Mukai K, Hiraoka I, Moriguchi S, Takama S, Kishino Y. Evaluation of the effect of arginine-enriched amino acid solution on tumor growth. J Parenter Enteral Nutr 1985; 9:428-434.
29. Brittenden J, Heys S D, Ross J, Park K G, Eremin O. Natural cytotoxicity in breast cancer patients receiving neoadjuvant chemotherapy: effects of L-arginine supplementation. Eur J Surg Oncol 1994; 20:467-472.
30. Heys S, Ogston K, Miller I, Hutcheon A, Walker L, Sarker T, Dewar J, Ah-See A, Eremin O. Potentiation of the response to chemotherapy in patients with breast cancer by dietary supplementation with L-arginine: results of a randomised controlled trial. Int J Oncol 1998; 12:221-225.
31. Federation of the American Society for Experimental Biology/Life Sciences Research Office (Anderson S A, Raiten D J, eds.) Safety of amino acids used as dietary supplements. Center for Food Safety and Applied Nutrition. 1992; FDA Contract No. 223-88-2124, Task No. 8.
32. Tangphao O, Chalon S, Moreno H, Hoffman B B, Blaschke T F. Pharmacokinetics of L-arginine during chronic administration in hypercholesterolemic patients. Clin Sci 1999; 96:199-209.
33. Mueller C. The regulatory status of medical foods and dietary supplements in the United States. Nutrition 1999; 15:249-251.
34. Mullen M J, Wright D, Donald A E, Thorne S, Thomson H, Deanfield J E: Atorvastatin but not L-arginine improves endothelial function in type I diabetes mellitus: a double-blind study. J Am Coll Cardiol 36:410-416, 2000.
35. Amir Lerman, MD; John C. Burnett, Jr, MD; Stuart T. Higano, MD; Linda J. McKinley, RN; David R. Holmes, Jr, MD, Long-term L-Arginine Supplementation Improves Small-Vessel Coronary Endothelial Function in Humans, Circulation. 1998; 97:2123-2128.
36. Bode-Böger S M, Muke J, Surdacki A, Brabant G, Böger R H, Frolich J C, Oral L-arginine improves endothelial function in healthy individuals older than 70 years, Vasc Med. 2003 May; 8(2):77-81.

1-38. (canceled)

39. An L-arginine-based formulation for oral absorption useful for dissolving fat plaques in arteries, said formulation comprising an L-arginine-containing compound in a concentration ranging from about 1% to about 80% (w/w);
wherein said formulation is characterized by odor levels less than 1 European Odor Units (ouE/m³) as measured by olfactometry; further wherein the edibility of said formulation is at least 4 according to the arginine scale.

40. The formulation according claim 39, wherein said formulation is characterized by pH of about 7 and, is palatable such that it is sustainable in a subject's mouth for at least 3 seconds allowing adsorption of at least 50% of said L-arginine content into said subject's blood system.

41. The formulation according claim 39, wherein said formulation is characterized by pH of about 7 and, is palatable such that it is sustainable in a subject's mouth for at least 3 seconds allowing adsorption of about 0.5 gr to about 100 gr of said L-arginine content into said subject's blood system.

42. The formulation according claim 39, wherein said formulation is capable of reducing nausea on a subject consuming at least 30 ml of said formulation per Kg weight of subject.

43. The formulation according claim 39, additionally comprising:
a. an L-arginine-containing compound in a concentration ranging from about 1% to about 80% (w/w);
b. acids having an LD₅₀ (oral rabbit) greater than 2 gm/kg;
c. organic solvents having an LD₅₀ (oral rabbit) greater than 2 gm/kg;
d. flavoring additives capable of altering the taste of said formulation as sensed by a subject which consumes said formulation with respect to said taste without said flavoring additives;
e. sequestrants consisting of materials having a log_{(Ki)} greater than 2 in which i represents the coordination number of the complex which is an integer between 1 and 4, and K represents the stability coefficient of said complex;
f. metabolizable preservatives; and,
g. water.

44. The formulation according claim 39, wherein at least one said L-arginine-containing compound is selected from the group consisting of L-arginine amino-acid, an oligo-peptide consisting of between two and ten L-arginine amino acids in which at least one amino-acid is L-arginine, a polypeptide consisting of at least ten amino-acids in which at least one amino-acid is L-arginine, derivatives of L-arginine which maintain the functionality of dissolving fat plaques in the artery, oligopeptides consisting of said derivatives of L-arginine, polypeptides consisting of said derivatives of L-arginine, and salts thereof.

45. The formulation according claim 39, further comprising at least one selected from a group consisting of (a) buffer adapted to maintain said pH value at about 7; (b) a sweetener; further wherein said sweetener is selected from the group comprising of sucrose, glucose, fructose, alkoxy aromatics, oximes, sulfamic acids, peptides, and succanilic acids, dihydro-chalcones and saccharin; (c) biocompatible emulsifiers, especially lecithin; (d) elements selected from a group consisting of Aspratic acid (Aspartate), Magnesium oxide (magnesia), Ginkgo Biloba, Chromium Picolinate, N-acetyl cysteine or any combination thereof; and any combination thereof.

46. The formulation according to claim 43, wherein at least one of said acids is selected from a group consisting of phosphoric acid, nicotinic acid, citric acid, acetic acid, maleic acid, propionic acid, butyric acid, tartaric acid, fumeric acid, adipic acid, and lactic acid and any combination thereof.

47. The formulation according claim 39, comprising at least one selected from (a) between 1% and 80% (w/w) L-arginine containing compound; (b) between 10% and 40% (w/w) L-arginine containing compound.

48. The formulation according claim 43, wherein at least one of said flavoring ingredient is selected from the group consisting of strawberry extract, pineapple extract, green tea leafs extract, anis extract, and combinations thereof; further wherein said preservatives are selected from the group consisting of benzoate salt, nipagine, nipazole, nitrate salt, nitrite salt, propionate salt, sulphite salt, sulphor dioxide and combinations thereof.

49. The formulation according to claim 39, further comprising at least one type of chromium complex; further wherein said chromium complex is chromium picolinate.

50. The formulation according to claim 39, especially used in mammals; further wherein especially useful for at least one selected from a group consisting of oral treatments, plaque removal, tooth bleaching, balancing blood pressure, bad breath, prevention of angina attacks, relieving intermittent claudication, improving endothelial function, sexual performance, sperm preparation or any combination thereof.

51. A tablet comprising the formulation of claim 39.

52. The tablet of claim 51, additionally comprising reagents selected from a group consisting of citric acid, citric esters, nipagine, nipazole, arginine, glycerin, anis extract, ethanol, ascorbic acid, strawberry extract, chromium picolinate, EDTA calcium salt, l-lysine and combinations thereof.

53. A method for treating cardiovascular disorders comprising steps:
a. obtaining an l-arginine-based orally-obtained useful for dissolving fat plaques in the artery characterized by odor levels lower than 1 European Odor Units (ouE/m³) as obtained by olfactometry and wherein said formulation is capable of inducing nausea on a subject consuming at least 30 ml of said formulation per Kg weight of subject comprising an L-arginine-containing compound in a concentration ranging from about 1% to about 80% (w/w); wherein said formulation having a pH of about 7 is palatable such that it is sustainable in a subject's mouth for at least 3 seconds allowing adsorption of at least 70% of said L-arginine content into said subject's blood system comprising:
i. an L-arginine-containing peptide comprising l-arginine in a concentration ranging from about 1% to about 80% (w/w);
ii. acids which have an LD₅₀ (oral rabbit) higher than 2 gm/kg;
iii. organic solvents, which have an LD₅₀ (oral rabbit) higher than 2 gm/kg;
iv. flavoring additives;
v. sequestrants consisting of materials having a log(Kᵢ)>2 in which i represents the coordination number of the complex which is an integer between 1 and 4, and K represents the stability coefficient of said complex;
vi. preservatives; and,
vii. water;
b. orally administering between about 5 ml to about 800 ml of said formula to a subject between once a week and five times a day; and,
c. repeating step (b) over a period of three days to twenty four months.

54. The method according to claim 53, additionally comprising at least one step selected from (a) selecting reagents from a group consisting of l-lysine and chromium complexes, especially chromium picolinate and combinations thereof; (b) selecting said reagents form of L-arginine-containing peptide from the group consisting of a monomer of l-arginine (amino acid), an oligopeptide consisting between two and ten l-arginine monomers, a polypeptide consisting of at least ten l-arginine, derivatives of l-arginine and oligopeptides; (c) selecting at least one of said organic acids from a group consisting of phosphoric acid, nicotinic acid, citric acid, acetic acid, maleic acid, propionic acid, butyric acid, tartaric acid, fumeric acid, adipic acid, and lactic acid and any combination thereof; (d) selecting said organic solvents from the group consisting of glycerin, glycerin derivatives, ethanol, phenol and combinations thereof; (e) selecting said flavoring ingredients from the group consisting of mint, peppermint, vanilla, chocolate, strawberry extract, pineapple extract, green tea leafs extract, anis extract, and combinations thereof, and consist between 0.1% and 5% of the total weight of the formulation; and polypeptides thereof.

55. The method according to claim 53, additionally comprising step of selecting said sequestrants from the group consisting of EDTA, EDTA salts, especially EDTA calcium salt and EDTA sodium salt.

56. The method according to claim 53, additionally comprising step of selecting said flavoring ingredients from the group consisting of strawberry extract, pineapple extract, green or black tea leafs extract, anis extract, and combinations thereof.

57. The method according to claim 53, additionally comprising step of selecting said preservatives from the group consisting of benzoates, nitrates, nitrites, propionates, sulphites, sulphor dioxide and combinations thereof.

58. The method according to claim 53, wherein said formulation further comprising biocompatible emulsifiers, especially lecithin.

59. The method according to claim 53, wherein said treatment is given prophylactically.

60. A method for producing an L-arginine-based orally-obtained formulation, comprising steps of:
a. mixing Arginine with Glycerin whilst heating said mixture with water;
b. neutralizing the acidity of said mixture by adding Citric Acid;
c. adding stabilizing egents selected from a group consisting of Nipagine and Nipazole or any combination thereof.

61. The method for producing an L-arginine-based orally-obtained formulation according to claim 60, additionally comprising at least one step of selected from (a) adding flavorings selected from a group consisting of Lemon, Strawberry, Orange, Mandarin or any combination thereof; (b) providing said L-arginine-based orally-obtained formulation with elements selected from a group consisting of Aspratic acid (Aspartate), Magnesium oxide (magnesia), Ginkgo Biloba, Chromium Picolinate, N-acetyl cysteine or any combination thereof; and any combination thereof.

62. The formulation according claim 39, comprises at least one ingredient, adapted to prevent any damage the synthesis of amino-acids and their derivatives, namely L-lysine.

63. The formulation according claim 39, comprising an appropriate amount of amino-acids and their derivatives, namely L-lysine.
